(19) United States
(12) Patent Application Publication
(10) Pub. No.: US 2022/0364039 A1
(43) Pub. Date: Nov. 17, 2022

(54) METHOD AND KIT FOR VESSEL FORMATION USING SMS STEM CELL-PRODUCED ECM AND SUBSTRATES

(71) Applicant: SMSBIOTECH, INC., San Marcos, CA (US)

(72) Inventor: Abdulkader Rahmo, San Marcos, CA (US)

(21) Appl. No.: 17/766,164

(22) PCT Filed: Sep. 29, 2020
(86) PCT No.: PCT/US20/53229

§ 371 Date: Apr. 1, 2022

Related U.S. Application Data
(60) Provisional application No. 62/912,412, filed on Oct. 8, 2019.

(51) Int. Cl. C12M 1/42; C12M 1/00

(52) U.S. Cl. CPC C12M 35/08; C12M 23/20; C12N 2533/90; C12N 5/069

(57) ABSTRACT

Disclosed herein are methods of inducing endothelial cell reorganization or differentiation to form micro- and macrovessel structures using an extracellular matrix, such as one derived from small mobile stem (SMS) cells, and a substrate, which can also be coated in molecules or otherwise physically manipulated to cause localized effects on reorganization Also disclosed is a kit implementation for performing endothelial cell reorganization.

### FIELD OF THE INVENTION

Aspects of the present disclosure relate generally to methods of inducing endothelial reorganization and differentiation to form micro- and macrovessel structures, such as tubules, using an extracellular matrix, such as one derived or obtained from a culture of small mobile stem (SMS) cells, which is contacted with a substrate in the presence of a cell population having endothelial cells. The disclosure also relates generally to a kit for implementation of the aforementioned process, which can be utilized to evaluate endothelial cell reorganization and differentiation in the presence or absence of a compound or drug, such as a molecule that induces, inhibits, or modulates tubule formation, angiogenesis, or arteriogenesis.

### BACKGROUND OF THE INVENTION

Endothelial cells make up the inner surface of blood vessels in vertebrates, including humans. These cells serve critical roles as a barrier for fluid, molecular, and cellular transport in and out of the bloodstream and in inflammation, clotting, blood pressure homeostasis, and angiogenesis. The vascular barrier is maintained by a monolayer of endothelial cells that are closely linked together through tight junctions, adherens junctions, and gap junctions. The endothelial monolayer is further stabilized by interactions with an underlying basement membrane, an extracellular matrix (ECM) secreted mainly by the endothelial cells and composed of collagen IV, laminin, nidogens, elastin, and proteoglycans. Attachment of endothelial cells to this ECM is important for migratory functions, including angiogenesis, wherein reorganization of endothelial cells into tube or tubule structures give rise to mature blood vessels. The necessity of this basement membrane ECM for reorganization is marked: while endothelial cells will eventually form tube structures after 1 week or up to 6-12 weeks depending on cell type, growth on reconstituted basement membrane material greatly accelerates tube formation on the order of hours and is completed within 1 day (Kubota et al. (1988) J Cell Biol. Oct;107(4) :1589-98).

Basement membrane ECM preparations such as Matrigel®, Which is secreted by Engelbreth-Holm-Swarm (EHS) mouse sarcoma cells, are commonly used in cell culture systems to mimic the extracellular environment in biological tissue. Within these matrices, endothelial cells adopt a flat, polygonal morphology characteristic of a monolayer endothelium and form capillary-like structures, a phenomenon that can be quantified in a “tube formation assay” (Kubota et al. (1988) J Cell Biol. Oct;107(4) :1589-98). Other useful applications include maintaining stem and precursor cells in an undifferentiated state during cell culture and inducing reorganization and differentiation of epithelial, endothelial and smooth muscle cells in vitro. ECM preparations are also used to support 3D cell culture in vitro, such as for modeling tumor development. However, murine-sourced ECM preparations do not fully recapitulate human ECM, which may interfere with sensitive stem cell or cancer conditions and raise issues when used for clinical applications.

Small mobile stem (SMS) cells have recently been isolated and characterized, for example in WO 2014/200940, hereby expressly incorporated by reference in its entirety. SMS cells can be adherent cells having variable size but generally range from 4.5 to 5.5 μm in diameter by light microscopy, and are obtained from sources such as umbilical cord, peripheral blood, bone marrow, or solid tissue, Furthermore, SMS cells are highly mobile. SMS cells may be used to produce ECM and/or ECM proteins in tissue culture, as described in WO 2017/172638, hereby expressly incorporated by reference in its entirety. The ECM produced by SMS cells can be purified and used for downstream applications as a human cell-derived alternative to murine ECM preparations currently available on the market.

In vitro angiogenesis and formation of vessel or tube-like structures by endothelial cells have far-reaching medical and biotechnological utilities. The need for improved or alternative processes for efficient and robust endothelial cell reorganization and differentiation and vessel formation is manifest.

### SUMMARY OF THE INVENTION

The present disclosure relates generally to methods for rapidly and efficiently inducing endothelial cell reorganization or differentiation, and microvessel formation, such as tubules, by contacting a population of cells comprising endothelial cells, preferably human, with an extracellular matrix (ECM) or a mixture having an ECM protein, including ECM or a mixture having an ECM protein derived or obtained from small mobile stem (SMS) cells, with a substrate, which isolates or occludes a subpopulation of the cells comprising endothelial cells, e.g., by partially isolating or partially occluding the population of cells comprising endothelial cells and a kit, which facilitates implementation of this process.

In some embodiments, the ECM or ECM protein, which optionally can be included in a mixture of proteins or peptides, used in the process is produced by SMS cells in a culture vessel, for example, as described in WO 2017/172638, and purified or isolated from the SMS cells. In some alternatives, the ECM or ECM protein, which optionally can be included in a mixture of proteins or peptides, preferably derived or obtained from SMS cells, can be kept in the original culture vessel and separated from the SMS cells or the ECM or ECM protein, which optionally can be included in a mixture of proteins or peptides, preferably derived or obtained from SMS cells, can be provided in the absence of SMS cells e.g., the ECM or ECM protein, which optionally can be included in a mixture of proteins or peptides, can be prepared and stored in the absence of SMS cells e.g., by refrigeration or freezing and thawing. In some embodiments, the culture vessels contemplated are containers commonly used in a laboratory or manufacturing setting, such as a dish, plate, well, flask, bottle, chamber, channel, tube, vessel, multi-well plate, niche, bioreactor, or any another container that can support cell culture and which can be constructed from a metal, mineral, e.g, quartz, plastic, polymer, glass, or ceramic, or any combination thereof.

In some embodiments, the ECM or ECM protein, which optionally can be included in a mixture of proteins or peptides, preferably derived or obtained from SMS cells, is seeded with endothelial cells or a population of cells comprising endothelial cells, wherein the endothelial cells or population of cells comprising endothelial cells will grow within the 3D matrix in a manner, which mimics or resembles vascular tissue (e.g., tubule formation, angiogenesis, or arteriogenesis). In some embodiments, the endothelial cells or population of cells comprising endothelial cells are human in origin. In some of these embodiments, the endothelial cells are human umbilical vein endothelial cells (HUVECs), human microvascular endothelial cells (HMECs), human lung microvascular endothelial cells (HLMECs), human aortic endothelial cells (HAECs), human pulmonary artery endothelial cells (HPAECs), or human coronary artery endothelial cells (HCAECs). In some embodiments, the population of cells comprising endothelial cells include in addition to endothelial cells, such as those described above, stem cells, fibroblasts, keratinocytes, progenitor cells, neurons, karyocytes, myoblasts, myocytes, adipocytes, osteoblasts, osteocytes, osteoclasts, macrophages, or leukocytes or any combination thereof. In some embodiments, the cells are grown in a medium that promotes angiogenesis, arteriogenesis, tubule formation, or vessel formation. In some embodiments, the endothelial cells or population of cells comprising endothelial cells are grown to about 20%, 30%, 40%, 50%, 60%, 70%, 80%, 90%, or 100% confluency or to 20%, 30%, 40%, 50%, 60% 70%, 80%, 90%, or 100% confluency. Alternatively, the endothelial cells or population of cells comprising endothelial cells are allowed to grow in contact with the ECM or ECM protein, which optionally can be included in a mixture of proteins or peptides, preferably derived or obtained from SMS cells, for 0, 1, 3, 4, 5, 6, 7, 8, 9, 10, 11, 12, 13, 14, 15, 16, 17, 18, 19, 20, 21, 22, 23, 24, 36, or 48 hours or 3-12 days or any time point within the range defined by any two of the aforementioned numbers before adding the one or more substrates.

In some embodiments, the substrate that induces endothelial cell reorganization or differentiation is any physical object, preferably aseptic or sterile, that can be applied to or contacted with a layer of endothelial cells or a population of cells comprising endothelial cells in order to isolate, partially isolate, occlude, or partially occlude the contacted cells from external conditions, e.g. environmental gas concentrations, the growth medium or salts, nutrients or other molecules in the growth medium, and such a substrate is constructed of metal, mineral, or polymer e.g. quartz, plastic, rubber, glass, or ceramic, preferably borosilicate or soda lime glass. In some embodiments, the culture vessel is a 24-well plate, and the substrate is a commonly used borosilicate glass microscope coverslip or glass sheet of variable shapes or sizes or thicknesses, e.g., circular, rectangular, square, triangular, irregular, obtained by cracking away sections of a preformed coverslip. In sonic embodiments, one or more molecules or compounds are added to the growth medium or coated onto the culture vessel or the one or more substrates or any combination thereof. These molecules or compounds include hormones, sugars, nucleic acids, amino acids, peptides, proteins, lipids, antibiotics, growth factors, inhibitors, such as inhibitors of angiogenesis, agonists, antagonists, inducers, such as inducers of angiogenesis, toxins. mutagens, cytokines, differentiators, coating agents, such as poly-lysine, or metabolites, or any combination or fragment thereof, and may be, optionally covalently attached to the culture vessel or the one or more substrates or any combination thereof.

In some embodiments, the one or more substrates are applied to the surface of the ECM or ECM protein, which optionally can be included in a mixture of proteins or peptides, preferably derived or obtained from SMS cells, such that the ratio or percentage of surface area of the one or more substrates to the surface area of the ECM or ECM protein, which optionally can be included in a mixture of proteins or peptides, preferably derived or obtained from SMS cells, is between 0.001%-98% such as percentages 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98% or within a range defined by any two of the aforementioned percentages, such as 0.001%-0.01%, 0.001%-0.1%, 0.001%-1%, 0,001%-10%, 0.001%-20%, 0.001%-30%, 0.001%40%, 0.001%-50%, 0.001%-60%, 1%-20%, 1%-30%, 1%-40%, 1%-50%, 1%-60%, 2%-20%, 2%-30%, 2%-40%, 2%-50%, 2%-60%, 40%-60%, or 50%-60% or about 0.001%-0.01%, 0.001%-0.1%, 0.001%-1%, 0.001%-10%, 0.001%-20%, 0.001%-30%, 0.001%-40%, 0.001%-50%, 0.001%-60%, 1%-20%, 1%-30%, 1%-40%, 1%-60%, 2%-20%, 2%-30%, 2%-40%, 2%-50%, 2%-60%, 40%-60%, or 50%-60%. In some embodiments, the endothelial cells contacting the one or more substrates reorganize to form structures resembling microvessel capillary networks or tubule networks, such as polygonal shapes or structures.

Preferred aspects of the present invention relate to the following numbered alternatives:

1. A method of inducing or accelerating reorganization or differentiation of a population of cells comprising endothelial cells, comprising:
contacting an extracellular matrix (ECM) or ECM protein, preferably derived or obtained from small mobile stem (SMS) cells, optionally human, with said population of cells comprising endothelial cells in a culture vessel; and
adding one or more substrates to said population of cells comprising endothelial cells in contact with the ECM or ECM protein in the culture vessel, wherein the one or more substrates isolate or occlude or partially isolate or partially occlude the population of cells comprising endothelial cells from growth media or external conditions or both.

2. The method of alternative 1, wherein the population of cells comprising endothelial cells comprises stem cells, fibroblasts, keratinocytes, progenitor cells, neurons, karyocytes, myoblasts, myocytes, adipocytes, osteoblasts, osteocytes, osteoclasts, macrophages, or leukocytes or any combination thereof.

3. The method of alternatives 1 or 2, wherein the ECM is comprised of agrin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, tenascin, perlecan, sortilin-related receptor, tensin, titin, total protein, or any combination thereof or a fragment of any one or more of the aforementioned.

4. The method of any one of alternatives 1-3, wherein the ECM protein is selected from the group consisting of agrin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, tenascin, perlecan, sortilin-related receptor, tensin, titin, and total protein, or a fragment of any one or more of the aforementioned proteins.

5. The method of any one of alternatives 1-4, wherein the culture vessel is a dish, plate, well, flask, bottle, chamber, channel, tube, niche, bioreactor, or another container that can support cell culture, which is, optionally, constructed from a metal, mineral, plastic, polymer, glass, or ceramic or any combination thereof.

6. The method of any one of alternatives 1-5, wherein the one or more substrates contacting the ECM or ECM protein in the culture vessel contact 1%, 2%, 5%, 10%, 15%, 70%, 25%, 30%, 35%, 40%, 45%, 50%, 55%, 60%, 65%, 70%, 75%, 80%, or any percentage within the range defined by any two of the aforementioned numbers of the total available surface area of the ECM or ECM protein in the culture vessel, preferably between 2%-60% or between about 2%-60%.

7. The method of any one of alternatives 1-6, wherein the percentage of surface area of the substrate, preferably a microscope coverslip, to occluding or partially occluding the ECM of ECM protein in the culture vessel is between 2%-60% or between about 2%-60%.

8. The method of any one of alternatives 1-7, wherein the population of cells comprising endothelial cells are contacted with the ECM in a growth medium suitable for angiogenesis, arteriogenesis, tubule, or vessel formation.

9. The method of alternatives 8, wherein the growth medium is M-25 medium supplemented with S-25 endothelial cell growth supplement at a concentration of between about 25:1 to about 75:1 or 25:1 to 75:1, preferably about 50:1 or 50:1.

10. The method of any one of alternatives 1-9, wherein the endothelial cells are primary endothelial cells or immortalized endothelial cells.

11. The method of any one of alternatives 1-10, wherein the endothelial cells are derived or obtained from a human.

12. The method of any one of alternatives 1-11, wherein the endothelial cells are human endothelial cells, such as human umbilical vein endothelial cells (HUVECs), human microvascular endothelial cells (HMECs), human lung microvascular endothelial cells (HLMECs), human aortic endothelial cells (HAECs), human pulmonary artery endothelial cells (HPAECs), or human coronary artery endothelial cells (HCAECs) or any combination thereof.

13. The method of any one of alternatives 1-12, wherein the population of cells are grown at 37° C. and in a humidified atmosphere comprised of 5% CO₂.

14. The method of any of alternatives 1-13, wherein the population of cells comprising endothelial cells are grown to about 20%, 30%, 40%, 50%, 60%, 70%, 80%, 90%, or 100% confluency or to 20%, 30%, 40%, 50%, 60%, 70%, 80%, 90%, or 100% confluency.

15. The method of any one of alternatives 1-14, wherein the population of cells comprising endothelial cells contacting the one or more substrates reorganize to form structures resembling microvessel capillary networks or tubule networks, such as polygonal shapes or structures.

16. The method of any one of alternatives 1-15, wherein the population of cells comprising endothelial cells are allowed to grow in contact with the ECM for 0, 1, 2, 3, 4, 5, 6, 7, 8, 9, 10, 11, 12, 13, 14, 15, 16, 17, 18, 19, 20, 21, 22, 23, 24, 36, or 48 hours or 3-12 days or any time point within the range defined by any two of the aforementioned numbers before adding the one or more substrates.

17. The method of any one of claims 1-16, wherein the population of cells comprising endothelial cells in contact with one or more substrates form tubules or vessel-like structures 0, 1, 2, 3, 4, 5, 6, 7 8, 9, 10, 11, 12, 13, 14, 15, 16, 17, 18, 19, 20, 21, 23, 24, 36, or 48 hours or 3-12 days or any time point within the range defined by any two of the aforementioned numbers after adding the one or more substrates.

18. The method of any one of alternatives 1-17, wherein the population of cells comprising endothelial cells in contact with the one or more substrates undergo 80%, 85%, 90%, 95?, or 100% reorganization within 1-5 days,

19. The method of any one of alternatives 1-18, wherein the one or more substrates comprise a metal, mineral, plastic, polymer, glass, or ceramic or any combination thereof, preferably soda lime glass or borosilicate glass.

20. The method of any one of alternatives 1-19, wherein the one or more substrates comprise borosilicate glass, preferably with a thickness of between 0.01-1 mm, such as a microscope slide coverslip.

21. The method of any one of alternatives 1-20, wherein one or more molecules or compounds are added to the growth medium or coated onto the culture vessel or the one or more substrates.

22. The method of alternative 21, wherein the one or more molecules or compounds comprise hormones, sugars, nucleic acids, amino acids, peptides, lipids, antibiotics, growth factors, inhibitors, such as inhibitors of angiogenesis, agonists, antagonists, inducers, such as inducers of angiogenesis, toxins, mutagens, cytokines, differentiators, coating agents, such as poly-lysine, or metabolites, or any combination or fragment thereof, optionally covalently attached to the culture vessel or the one or more substrates.

23. The method of alternative 21., wherein the one or more molecules or compounds comprise a protein, such as a growth factor, a cytokine, a peptide hormone, an antibody, a protein hormone, an extracellular matrix protein, an epidermal growth factor (EGF), a platelet derived growth factor (PDGF), a fibroblast growth factor (FGF and bFGF), a transforming growth factor (TGF-a and TGF-P 1, 2, & 3), a vascular endothelial growth factor (VEGF), a hepatocyte growth factor (HGF), a keratinocyte growth factor (KGF), a nerve growth factor (NGF), erythropoietin (EPO), an insulin-like growth factors (IGF-I and IGF-11), an interleukin cytokine (IL-la, IL-Ip, IL-2, IL-3, IL-4, IL-5, IL-6, IL-7, IL-8, IL-9, IL-10, IL-11, IL-12, IL-13), an interferon (IFNa, IFN-p, and IFN-y), a tumor necrosis factor (TNFa and TNF-p), a colony stimulating factor (GM-CSF and M-CSF), insulin, parathyroid hormone, fibronectin, collagenase, collagen, elastin, laminin, agrin, nidogen, entactin, coagulation factor XIII A chain, apolipoprotein E, anithrombin III, bone morphogenic protein 1, vitronectin, acidic leucine-rich nuclear phosphoprotein 32 family member A (ANP32A), calcineurin-like phosphoesterase, peptidyl-prolyl cis-trans isomerase, P-enolase, fermitin family homolog 1, microtubule-associated protein RP/EB (MAPREI), a heat shock protein, LIM and senescent cell antigen-like-containing domain protein 1 (LIMS 1), myosin regulatory protein, profilin-1, glycogen phosphorylase, flavin reductase, mitogen activated protein kinase, protein phosphatase, tubulin, chloride intracellular channel proteins, wings apartlike protein homolog (WAPAL), cell division control proteins, osteopontin, BPI fold containing, elongation factor, plasminogen, aldo-keto reductase, or keratin, or any combination or fragment thereof, optionally covalently attached to the culture vessel or the one or more substrates.

24. The methods of any one of alternatives 21-23, wherein the one or more molecules are coated in a specific pattern or shape on the one or more substrates.

25. The method of any one of alternatives 1-24, wherein the culture vessel or the one or more substrates are siliconized, optionally with a pattern or shape,

26. The method of any one of alternatives 1-25, wherein the culture vessel or the one or more substrates are etched or scratched, optionally with a pattern or shape.

27. The method of any one of alternatives 1-26, wherein the one or more substrates are coated in ECM or ECM protein, optionally derived or obtained from a population of SMS cells and, optionally in a pattern or shape.

28. A kit for inducing or accelerating reorganization or differentiation of a population of cells comprising endothelial cells, comprising:
one or more culture vessels comprising one or more regions coated with extracellular matrix (ECM) or ECM protein, preferably derived or obtained from a population of small mobile stem (SMS) cells, optionally human;
optionally, a growth medium; and
one or more substrates configured to fit within said one or more culture vessels.

29. The kit according to alternative 28, wherein the one or more culture vessels comprise a dish, plate, well, flask, bottle, chamber, channel, tube, niche, bioreactor, or another container that can support cell culture and comprises a metal, mineral, plastic, polymer, glass, or ceramic or any combination thereof.

30. The kit according to alternatives 28 or 29, wherein the ECM is comprised of agrin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, tenascin, perlecan, sortilin-related receptor, tensin, titin, or total protein, or any combination thereof or a fragment of any one or more of the aforementioned.

31. The kit according to any one of alternatives 28-30, wherein the ECM protein is selected from the group consisting of agrin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, tenascin, perlecan, sortilin-related receptor, tensin, titin, and total protein, or a fragment of any one or more of the aforementioned.

32. The kit according to any one of alternatives 28-31, wherein the one or more culture vessels comprise multi-well culture plates and the regions coated with ECM or ECM protein are the bottoms of each well of said one or more culture vessels.

33. The kit according to any one of alternatives 28-32, wherein the growth medium is suitable for angiogenesis, arteriogenesis, tubule, or vessel formation.

34. The kit according to any of alternatives 28-33, Wherein the growth medium is M-25 medium supplemented with S-25 endothelial cell growth supplement at a concentration of between about 25:1 to about 75:1 or 25:1 to 75:1, preferably about 50:1 or 50:1.

35. The kit according to any of alternatives 28-34, wherein the one or more substrates comprise a metal, mineral, plastic, polymer, glass, or ceramic or any combination thereof, preferably soda lime glass or borosilicate glass.

36. The kit according to any of alternatives 28-35, wherein the one or more substrates comprise borosilicate glass, preferably with a thickness of between 0.01-1 mm, such as a microscope slide coverslip.

37. The kit according to any of alternatives 28-36, wherein one or more molecules or compounds are added to the growth medium or coated onto the culture vessel or the one or more substrates.

38. The kit according to alternative 37, wherein the one or more molecules or compounds comprise hormones, sugars, nucleic acids, amino acids, peptides, lipids, antibiotics, growth factors, inhibitors, such as inhibitors of angiogenesis, agonists, antagonists, inducers, such as inducers of angiogenesis, toxins, mutagens, cytokines, differentiators, coating agents, such as poly-lysine, or metabolites, or any combination or fragment thereof, optionally covalently attached to the substrate.

39. The kit according to alternative 37, wherein the one or more molecules or compounds comprise a protein, such as a growth factor, a cytokine, a peptide hormone, an antibody, a protein hormone, an extracellular matrix protein, an epidermal growth factor (EGF), a platelet derived growth factor (PDGF), a fibroblast growth factor (FGF and bFGF), a transforming growth factor (TGF-a and TGF-P 1, 2, & 3), a vascular endothelial growth factor (VEGF), a hepatocyte growth factor (HGF), a keratinocyte growth factor (KGF), a nerve growth factor (NGF), erythropoietin (EPO), an insulin-like growth factors (IGF-I and IGF-11), an interleukin cytokine (IL-la, IL Ip, 1L-2, IL-3, II, 4, IL-5, IL-6, IL-7, TL-8, IL-9, IL-10, IL-11, IL-12, IL-13), an interferon (IFNa, IFN-p, and IFN-y), a tumor necrosis factor (TNFa and TNF-p), a colony stimulating factor (GM-CSF and M-CSF), insulin, parathyroid hormone, fibronectin, collagenase, collagen, elastin, laminin, agrin, nidogen, entactin, coagulation factor XIII A chain, apolipoprotein E, anithrombin III, bone morphogenic protein 1, vitronectin, acidic leucine-rich nuclear phosphoprotein 32 family member A (ANP32A), calcineurin-like phosphoesterase, peptidyl-prolyl cis-trans isomerase, P-enolase, fermitin family homolog 1, microtubule-associated protein RP/EB (MAPRE1), a heat shock protein, LIM and senescent cell antigen-like-containing domain protein 1 (LIMS 1), myosin regulatory protein, profilin-1, glycogen phosphorylase, flavin reductase, mitogen activated protein kinase, protein phosphatase, tubulin, chloride intracellular channel proteins, wings apartlike protein homolog (WAPAL), cell division control proteins, osteopontin, BPI fold containing, elongation factor, plasminogen, aldo-keto reductase, or keratin, or any combination or fragment thereof, optionally covalently attached to the substrate.

40. The kit of any of alternatives 37-39, wherein the one or more molecules are coated in a specific pattern or shape on the one or more substrates.

41. The kit of any of alternatives 28-40, wherein the culture vessel or the one or more substrates are siliconized.

42. The kit of any of alternatives 28-41, wherein the culture vessel or the one or more substrates are etched or scratched, optionally in a pattern or shape.

43, The kit of any of alternatives 28-42, wherein the one or more substrates are coated in ECM or ECM protein, optionally derived or obtained from a population of SMS cells and, optionally in a pattern or shape,

44. The kit according to any of alternatives 28-43, wherein the one or more culture vessels comprise a. growth medium, and the one or more substrates are sterile and are packaged to maintain sterility until use, e.g., sealed in plastic film or wrap, and optionally vacuum packed.

45. The kit of any one of alternatives 28-44, wherein the one or more substrates contacting the ECM or ECM protein in the culture vessel contact 1%, 2%, 5%, 10%, 15%, 20%, 25%, 30%, 35%, 40%, 45%, 50%, 55%, 60%, 65%, 70%, 75%, 80%, or any percentage within the range defined by any two of the aforementioned numbers of the total available surface area of the ECM or ECM protein in the culture vessel, preferably between 2%-60% or between about 2%-60%.

46. The kit of any one of alternatives 28-45, wherein the percentage of surface area of the substrate, preferably a microscope coverslip, which occludes or partially occludes the surface area of the ECM or ECM protein in the culture vessel is between 2%-60% or between about 2%-60%.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts differentiation and reorganization of endothelial cells when a substrate, here a borosilicate glass coverslip, is applied on top. Reorganization starts at the edges of the substrate and proceeds inwards between the surfaces of the culture vessel and substrate.

FIG. 2 depicts further progression over time of differentiation and reorganization of endothelial cells underneath the substrate.

FIG. 3 depicts obvious flattening and vacuolization of endothelial cells underneath the substrate as compared to the subpopulation that is uncovered.

FIG. 4 depicts flattening, vacuolization, and polygonal reorganization of endothelial cells underneath the substrate.

FIG. 5 depicts flattening, vacuolization, and polygonal reorganization of endothelial cells underneath the substrate.

FIG. 6 depicts the same flattening, vacuolization, and polygonal reorganization of endothelial cells underneath an irregularly shaped substrate. Arrow 1 denotes the edge of the substrate, here an irregularly shaped borosilicate glass coverslip. Arrow 2 denotes an organized microvessel structure.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length,

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In some embodiments, the “purity” of any given agent (e.g., antibody, polypeptide binding agent) in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used herein, the terms “function” and “functional” and the like refer to a biological, enzymatic, or therapeutic function.

The term “isolated” is meant material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The practice of the present disclosure will employ, unless indicated specifically to the contrary, conventional methods of molecular biology and recombinant DNA techniques within the skill of the art, many of which are described below for the purpose of illustration, Such techniques are explained fully in the literature. See, e.g., Sambrook, el al, Molecular Cloning A Laboratory Manual (3^{rd} Edition, 2000); DNA Cloning: A Practical Approach, vol. 1 & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Flames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken N.J., John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005).

Embodiments disclosed herein relate to compositions comprising an ECM or ECM protein, which optionally can be included in a mixture of proteins or peptides, preferably derived or obtained from SMS cells, optionally human, which may or may not comprise one or more induced cross-links, which have been induced by exposure to a cross-linking agent or a cross-linking technique e.g., radiation, chemical, mechanical, or temperature.

“SMS cells” as used herein refers to a cell or a cell population characterized in that the cells are adherent cells of about 5 μm in diameter. The SMS cells are equi-dimensional, with strict radial symmetry, and exhibit a translucent cytoplasm and circular nucleus that includes a centrally located circle of a different light contrast, as viewed in a light microscope. In addition, SMS cells demonstrate an extraordinary resistance to various non-physiological conditions, including low and high temperature, freezing and thawing in standard growth medium, dehydration, high pH values, and variations of ionic strength. SMS cells are also characterized by their high mobility of up to about 1.5 μm/sec. SMS cells are cultured in either a suspension or adherent state, and ECM isolated from the SMS cells as previously described, for example in WO 2017/172638.

“Extracellular matrix (ECM)” as used herein is an extracellular component consisting of an intricate network of ECM proteins and polysaccharides that are secreted by cells. SMS-derived ECM and ECM proteins refers to ECM and ECM protein that has been produced by, derived from, isolated from, or otherwise obtained from a culture of SMS cells. In some embodiments, the ECM or ECM protein, which may have been isolated from a culture of SMS cells, comprises agrin, filaggrin, mucin, secreted phosphoprotein 24 (bone matrix), nidogen, cadherins, clathrin, collagen, defensin, elastin, entactin, fibrillin, fibronectin, vitronectin, keratin, laminin, microtubule-actin cross-linking factor I , SPARC-like protein, nesprin (nesprin-1, nesprin-2, nesprin-3), fibrous sheath-interacting protein, myomesin, nebulin, keratinocyte proline rich protein, plakophilin, integrin, talins, exportins, transportin, tenascin, perlecan, sortilin-related receptor, tensin, titin, total protein, or a fragment of any one or more of the aforementioned proteins. In some embodiments, the one or more ECM protein is modified. In some embodiments, a modified ECM protein is modified by denaturation, acetylation, acylation, carboxylation, glycosylation, hydroxylation, lipidation, methylation, pegylation, phosphorylation, prenylation, sulfation, and/or ubiquitination. In some embodiments, a variety of cells is grown with and interacts with SMS-derived ECM. In some embodiments, cells including but not limited to endothelial cells, stem cells, fibroblasts, keratinocytes, progenitor cells, neurons, karyocytes, myoblasts, myocytes, adipocytes, osteoblasts, osteocytes, osteoclasts, macrophages, or leukocytes are grown in the presence of SMS-derived ECM and/or interact with SMS-derived ECM.

In some embodiments, endothelial cells are grown with and interact with SMS-derived ECM. In some embodiments, the endothelial cells migrate into the SMS-derived ECM. In some embodiments, the endothelial cells actively proliferate within the SMS-derived ECM. In some embodiments, endothelial cells differentiate into tubes and survived in that state for 1 day, 2 days. 3 days, 4 days, 5 days, 6 days, 1 week, 2 weeks, 3 weeks, 4 weeks, 1 month, 2 months, 3, months, 4 months, 5 months, 6 months, 9 months, 1 year, or longer, or a duration within a range defined by any two of the aforementioned durations. In some embodiments, the endothelial cells formed transiently aligned cells along the edges of aligned. SMS cells with endothelial cells largely absent at the core between borders, which is reminiscent of larger tubular (vessel) structures.

In some alternatives, the ECM is also decellularized (e.g., by using a chemical, physical, and/or enzymatic approach). Preferably, a decellularization approach is configured such that the ECM scaffold maintains its structural and chemical integrity. In addition, various molecular components of the SMS derived ECM can be enriched or isolated. ECM in various tissues of various organs can be shown to be similar or identical to ECM from in vitro SMS cell culture. The ECM produced by the aforementioned SMS cells may be freeze dried into powder and stored as such. ECM in powder or other form is capable of being used for various applications such as promoting cell growth or cell differentiation in vitro (such as for 3D cell culture) or in vivo (such as in promoting wound healing) (or inhibiting tumor growth).

SMS cells are also grown on a variety of culture vessels in some alternatives. SMS cells can be grown on, e.g., a dish, plate, well, flask, bottle, chamber, channel, tube, vessel, multi-well plate, niche, bioreactor, or any another container that can support cell culture and which are constructed from a metal, mineral, e.g, quartz, plastic, polymer, glass, or ceramic, or any combination thereof. The surface of the dish, plate, well, flask, bottle, chamber, channel, tube, vessel, multi-well plate, niche, bioreactor, or any other container that can support cell culture can be pretreated with etched or scratched surfaces of geometric shapes. The surface of these culture vessels may be pretreated with a chemical or physical treatment, including, for example, etching, scratching, the use of borosilicate, silicone (siliconized), silanization, mechanical abrasion, blasting, silicon carbide, solvent, acid, anodizing, or the application of a carbon film such as carbon evaporation e.g., on a desired geometric shape or any combination thereof The pretreatment can provide a geometric shape or greater porosity on the surface of the container e.g. a dish, plate, well, flask, bottle, chamber, channel, tube, vessel, multi-well plate, niche, bioreactor, or any other container that can support cell culture. The geometric shape can include, for example, one or more of a line, curve, web, groove, ridge, or other shape. SMS cells deposited or introduced to the pretreated dish, plate, well, flask, bottle, chamber, channel, tube, vessel, multi-well plate, niche, bioreactor, or any other container that can support cell culture organize about the geometric shape, using the shape as a guide for the organization of cell culture growth and for depositing ECM.

In some embodiments, cells or a population of cells, including but not limited to SMS cells, endothelial cells, stem cells, fibroblasts, keratinocytes, progenitor cells, neurons, karyocytes, myoblasts, myocytes, ad ipocytes, osteoblasts, osteocytes, osteoclasts, macrophages, or leukocytes, are cultured in a culture vessel in a growth medium containing components necessary to sustain biological life, but also may include other components to enhance, accelerate, induce or inhibit growth rate or other cellular properties, behaviors, or functions, e.g. any molecule, compound, peptide, protein, hormone, inducer, inhibitor, growth factor or substance described or suggested herein. An example growth medium is high sugar basal medium (Dulbecco's Modified Eagle Medium (DMEM), [+] 6 g/L D-glucose, [−] sodium pyruvate, [−] L-glutamine, [−] Phenol red), to which 1% GlutaMAX™-I (100×), 10% calf serum, and 5 μg/mL human insulin was added. Alternatively, a medium not containing any calf serum can be used. In some embodiments, M-25 medium with S-25 supplement is used. As used herein, M-25 medium is a growth medium optimized for endothelial cell growth and includes, but is not limited to, glycine, alanine, arginine, asparagine, aspartic acid, cysteine, glutamic acid, glutamine, histidine, isoleucine, leucine, lysine, methionine, phenylalanine, proline, serine, threonine, tryptophan, tyrosine, valine, choline, pantothenic acid, folinic acid, myo-inositol, niacinamide, pyridoxal, riboflavin, thiamine, vitamin B12, biotin, ammonium metavanadate, ammonium molybdate, calcium chloride, cupric sulfate, ferric sulfate, magnesium sulfate, manganese sulfate, nickelous chloride, potassium phosphate, sodium bicarbonate, sodium chloride, sodium metasilicate, sodium selenite, tin chloride, zinc sulfate, adenine, glucose, alpha-lipoic acid, HEPES, phenol red, putrescine, sodium pyruvate, thymidine, or any combination thereof. As used herein, S-25 supplement is a growth medium supplement that is added to enhance endothelial cell growth and includes, but is not limited to, fetal bovine serum, hydrocortisone, human epidermal growth factor, basic fibroblast growth factor, heparin, ascorbic acid, or any combination thereof. The cells can be suspended occasionally by swirling. Cells are commonly, but not necessarily, grown in a humidified atmosphere containing 5% CO₂ at 37° C.

In some embodiments described herein, endothelial cells can be primary (taken directly from a living tissue sample and having a limited capacity for cellular replication, otherwise known as the “Hayflick limit”), or immortalized (naturally or artificially mutated, induced, or otherwise adapted to have cancerous or stem cell-like properties that allow cells to be proliferated in vitro indefinitely or for extended periods of time), and can be human in origin. Human endothelial cells useful in the systems described herein can be obtained from all organs and tissues, e.g, aorta, artery, bladder, bone, brain, carotid artery, colon, coronary artery, dermis, kidney, liver, lung, mammary gland, muscle, ovary, pancreas, placenta, prostate, pulmonary artery, pulmonary vein, retina, small intestine, spleen, stomach, testis, thymus, thyroid, umbilical cord, uterus, vein, and vena cava, and include, but are not limited to, human umbilical vein endothelial cells (HUVECs), human microvascular endothelial cells (HMECs), human lung microvascular endothelial cells (HLMECs), human aortic endothelial cells (HAECs), human pulmonary artery endothelial cells (HPAECs), or human coronary artery endothelial cells (HCAECs).

In some embodiments, endothelial cells or a population of cells containing endothelial cells are allowed to grow in contact with the ECM or ECM protein, which optionally can be included in a mixture of proteins or peptides, preferably derived or obtained from SMS cells, for 0, 1, 2, 3, 4, 5, 6, 7, 8, 9, 10, 11, 12, 13, 14, 15, 16, 17, 18, 19, 20, 21, 22, 23, 24, 36, or 48 hours or 3-12 days or any time point within the range defined by any two of the aforementioned numbers. In some embodiments, endothelial cells or a population of cells containing endothelial cells are grown to about 20%, 30%, 40%, 50%, 60%, 70%, 80%, 90%, or 100% confluency or to 20%, 30%, 40%, 50%, 60%, 70%, 80%, 90%, or 100% confluency.

As used herein, the term “substrate” refers to any physical object, preferably aseptic or sterile, that can be applied to a layer of endothelial cells or a population of cells containing endothelial cells in order to isolate, partially isolate, occlude, or partially occlude the contacted cells from external conditions, e.g. environmental gas concentrations, the growth medium or salts, nutrients or other molecules in the growth medium. As described in the examples and show in the figures, the act of applying a substrate to endothelial cells or a population of cells comprising endothelial cells grown in the ECM or ECM protein, which optionally can be included in a mixture of proteins or peptides, preferably derived or obtained from SMS cells, optionally human, induces endothelial cell reorganization, tubule formation, angiogenesis, arteriogenesis, or microvessel formation or any combination thereof In some embodiments, the substrate is constructed of or comprises a metal, mineral, e.g. quartz, plastic, polymer, glass, or ceramic or any combination thereof. In some embodiments, the substrate comprises borosilicate glass, such as a microscope coverslip or glass sheet and can be of variable shapes and sizes, e.g., circular, rectangular, square, triangular, irregular, obtained by cracking away sections of a preformed coverslip, and thicknesses, e.g. 0.01-1, 0.085-0.13 (#0), 0.13-0.16 (#1), 0.16-0.19 (#1.5) 0.17-0.18 (#1.5H), 0.19-0.23 (#2), 0.25-0.35 (#3), 0.43-0.64 (#4) mm in thickness. In some embodiments, one or more substrates can be used per contiguous ECM or ECM protein surface in the culture vessel, and the one or more substrates occupy a percentage of the total surface area of ECM or ECM protein between 0.001%-98% such as percentages 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98% or within a range defined by any two of the aforementioned percentages, such as 0.001%-0.01%, 0.001%-0.1%, 0.001%-1%, 0.001%-10%, 0.001%-20%, 0.001%-30%, 0.001%-40%, 0.001%-50%, 0.001%-60%, 1%-20%, 1%-30%, 1%-40%, 1%-50%, 1%-60%, 2%-20%, 2%-30%, 2%-40%, 2%-50%, 2%-60%, 40%-60%, or 50%-60% or about 0.001%-0.01%, 0.001%41%, 0.001%-1%, 0.001%-10%, 0.001%-20%, 0.001%-30%, 0.001%-40%, 0.001%-50%, 0.001%-60%, 1%-20%, 1%-30%, 1%-40%, 1%-50%, 1%-60%, 7%-20%, 2%-30%, 7%-40%, 2%-50%, 2%-60%, 40%-60%, or 50%-60%. In some embodiments, the endothelial cells contacting the one or more substrates reorganize to form structures resembling microvessel capillary networks or tubule networks, such as polygonal shapes or structures. Each of the one or more substrates can occupy a different percentage of the total surface area of the ECM or ECM protein, inducing reorganization and differentiation of the endothelial cells or the population of cells comprising endothelial cells in different regions of the surface of the ECM or ECM protein in the culture vessel.

Similarly to the culture vessel, the substrates can be pretreated or etched or scratched surfaces, optionally having geometric shapes or patterns. The surface of these substrates may be pretreated with a chemical or physical treatment, including, for example, etching, scratching, the use of borosilicate, silicone (siliconized), silanization, mechanical abrasion, blasting, silicon carbide, solvent, acid, anodizing, or the application of a carbon film such as carbon evaporation e.g., on a desired geometric shape. The pretreatment can provide a geometric shape or pattern or greater porosity on the surface of the substrate. The geometric shape or pattern can include, for example, one or more of a line, curve, web, groove, ridge, or other shape. Endothelial cells contacted to the pretreated substrate organize about the geometric shape, using the shape as a guide for reorganization and microvessel or tubule formation.

In some embodiments, the substrates or culture vessels are coated in one or more molecules or compounds, including but not limited to proteins, hormones, sugars, nucleic acids, amino acids, peptides, lipids, antibiotics, growth factors, inhibitors, such as inhibitors of angiogenesis, agonists, antagonists, inducers, such as inducers of angiogenesis, toxins, mutagens, cytokines, differentiators, coating agents or metabolites. These molecules or compounds may be covalently linked to the substrate or culture vessel material directly, e.g. silanization, or through an intermediate coating, e.g, poly-lysine, polyacrylamide, or agarose, or to the ECM or ECM protein by crosslinking, e.g. by radiation, chemical, mechanical, or temperature. In some alternatives, the one or more molecules or compounds can be supplemented in the growth medium. In some embodiments, one or more of the aforementioned molecules or compounds may be present in an amount of 0.000001, 0.00001, 0.0001, 0.001, 0.01, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10%, or an amount within a range defined by any two of the aforementioned values. In some embodiments, one or more of the aforementioned molecules or compounds may be present in an amount of 0.00000001, 0.0000001, 0.000001, 0.00001, 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 mM or an amount within a range defined by any two of the aforementioned values.

A single contiguous surface of ECM or ECM protein seeded with endothelial cells or a population of cells comprising endothelial cells is not limited to a single coverslip or substrate. Each of the one or more coverslips or substrates can occupy a different percentage of the total surface area of the ECM or ECM protein, inducing reorganization and differentiation of the endothelial cells in different regions of the surface of the ECM or ECM protein in the 24-well plate or other culture vessel. Coating of the one or more substrates with one or more molecules or compounds, including but not limited to proteins, hormones, sugars, nucleic acids, amino acids, peptides, lipids, antibiotics, growth factors, inhibitors, such as inhibitors of angiogenesis, agonists, antagonists, inducers, such as inducers of angiogenesis, toxins, mutagens, cytokines, differentiators, coating agents or metabolites in patterns, shapes, or micropatterns, optionally covalently linked to the substrate either directly or through an intermediate, can also be used to subject different localized subpopulations of endothelial cells or population of cells comprising endothelial cells and to create controlled areas of micro- or macro-heterogeneity within the cell population.

As used herein, the term “hormones” refer to molecules and compounds, either natural or man-made, that induce changes in cell property, behavior, or function through signal transduction pathways. Possible human hormones include, but are not limited to, nitric oxide, epinephrine, melatonin, noreprinephrine, triiodothyronine, thyroxine, prostaglandins, leukotrienes, prostacyclin, thromboxane, amylin, adiponectin, adrenocorticotropic hormone, angiotensinogen, angiotensin, antidiuretic hormone, atrial-natriuretic peptide, brain natriuretic peptide, cal citonin, cholecystokinin, corticotropin-releasing hormone, cortistatin, encephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, growth hormone, inhibin, insulin, insulin-like growth factor, leptin, lipotropin, leuteinizing hormone, melanocyte stimulating hormone, motilin, orexia, osteocalcin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone, thyrotropin-releasing hormone, vasoactive intestinal peptide, guanylin, uroguanylin, testosterone, aldosterone, estradiol, cortisol, progesterone, calicitriol, or calcidiol.

As used herein, “inducers” refer to substances that cause a particular change in cellular property, behavior, or function, whereas “inhibitors” refer to substances that prevent or delay a particular change in cellular property, behavior, or function despite the presence of a stimulus that would otherwise cause such a change. In some embodiments, inducers and inhibitors affect endothelial cell growth, replication, motility, adherence, permeability, tube or vessel formation, angiogenesis, RNA or protein expression, uptake, excretion, apoptosis, necrosis, or other cellular property or function either as isolated cells or within vascular or musculovascular tissue, and may include hormones listed herein. These inducers and inhibitors include, but are not limited to, nitric oxide, adrenocorticotropic hormone, adrenomedullin, angiotensin, bradykinin, calcitonin, endothelin, erythropoietin, gastrin, gonadotropins, growth hormone, insulin, insulin-like growth factor, leptin, neuropeptide Y, oxytocin, parathyroid hormone, relaxin, thrombopoietin, tyroid-stimulating hormone, vasopressin, kininogen, ghrelin, somatostatin, vasoinhibins, adiponectin, corticotropin-releasing hormone, vascular endothelial growth factor (VEGF), angiopoietin-1, angiopoietin-2, basic fibroblast growth factor (bFGF), corneal endothelium modulation factor (CEMF), platelet-derived growth factor (PDGF), epidermal growth factor (EGF), bone morphognenetic protein 4 (BMP4), Indian hedgehog (IHH), phthalimide neovascular factor 1, retinol, retinoic acid, semaphorins, ephrins, transforming growth factor beta (TGF-β), tricyclodecan-9-yl-xanthogenate, 8,9-dihydroxy-7-methylbenzo(b)quinolizinium, prostacyclin, afatinib, axitinib, bevacizumab, cabozantinib, everolimus, lenalidomide, lenvatinib mesylate, motesnaib, nintedanib, pazopanib, ponatinib, ramucirurmab, regorafenib, sorafenib, sunitinib, thalidomide, vandetanib, ziv-aflibercept, and L-DOPA. As used herein, the terms “growth factors” are molecules, compounds, peptides, or proteins that promote growth in cells, and “cell differentiators” are molecules, compounds, peptides, or proteins that induce differentiation in cells, such as stem cells and endothelial cells, and may include, but are not limited to, any substance, molecules, compounds, peptide, or proteins listed thereof, or elsewhere herein, or those substances described in WO 2017/172638, herein expressly incorporated by reference in its entirety.

As used herein, the term “antibiotics” are any substance used to kill or inhibit the growth of cells or microorganisms. In some embodiments, antibiotics include, but are not limited to, antibacterial, antimycotic, antifungal, anthelmintic, and antiparasitic molecules or compounds. In other alternatives, antibiotics refer to molecules or compounds that kill or inhibit the growth of eukaryotic cells, including, but not limited to, blasticidin S, bleomycin, doxycycline, geneticin, hygromycin B, phleomycin, mycophenolic acid, neomycin, puromycin, or tetracycline. As used herein, the term “toxins” refer to any substance, generally of biological origin, used to kill or inhibit the growth of cells, sometimes by causing cellular damage, and include, but are not limited to, cyanotoxins, dinotoxins, neurotoxins, neurotoxins, myotoxins, and cytotoxins. As used herein, the term “mutagens” refer to any substance that causes modifications or mutations in the genetic material of a cell and include, but are not limited to, radioactive compounds, reactive oxygen species, deaminating agents, polycyclic aromatic hydrocarbons, alkylating agents, e.g. N-ethyl-N-nitrosourea, nucleotide analogs, e.g. 5-bromouracil, and intercalating agents, e.g. doxorubicin.

As used herein, the term “coating agents” refer to any compound that are applied to the surface of any object to impart certain physical, chemical, and biological properties, e.g. hydrophilicity, hydrophobicity, transparency, opacity, light reflection or absorption, adhesion, adsorption, electrical conductivity or resistance, static charge capacity, stiffness, porosity, roughness, cleanliness, chemical reactivity or inertness, cellular attachment, enzymatic activity, or biological molecule display. In some embodiments, the substrates or culture vessels are coated in, e.g., poly-lysine, poly-ornithine, collagen, gelatin, fibronectin, vitronectin, or laminin to enhance cell attachment. In other embodiments, the substrates or culture vessels are siliconized with, e.g., dimethyldichlorosilane, to increase hydrophobicity and limit cell attachment.

As used herein, the term “stiffness” refers to the ability of a material to resist elastic deformation in response to a load. This property of the material is quantified as the elastic modulus, or Young's modulus, and is measured in units of pressure, e.g., pascals. Biological tissues exhibit elastic moduli on the order of kilopascals and this stiffness is partially dictated by the composition of an ECM support, if present (Handorf et al. (2015) Organogenesis. Jan;11(1): 1-15). Consequently, in some embodiments, the stiffness of the culture vessel surface on which the endothelial cells or population of endothelial cells are seeded can be manipulated by modifying the ECM composition or constituent ECM proteins. In further embodiments, the stiffness of the substrate can be similarly varied through an ECM coating or the aforementioned coating agents, such as silicone wherein the thickness or composition of silicone deposited by siliconization determines the stiffness of the surface contacting cells. A skilled person in the art would appreciate that it is routine and conventional to apply silicone layers in varying thicknesses or compositions to achieve a certain stiffness, and quantify the elastic modulus through methods such as atomic force microscopy, deformation by stretching or spherical indentation, or rheology (Bashirzadeh et al. (2018) J Vis Exp. Jul 3;(137)). In some embodiments, this stiffness can be designed to mimic biological tissues ranging from bone (15-20 kPa) to fat tissue (0.5-1 kPa) (Handorf et al. (2015) Organogenesis. Jan;11(1): 1-15). By altering surface stiffness, contacted cells exhibit desired properties and functions as a response to the exerted mechanical forces.

As used herein, the term “metabolites” are any molecules or compounds that are produced by a chemical, biological, or enzymatic reaction or any intermediate molecules or compounds of said chemical, biological, or enzymatic reaction. Metabolites may have functions or effects on enzymes or cells different from the original molecule or compound from which they are derived. :Metabolites include, but are not limited to, amino acids, organic acids, nucleic acids, fatty acids, lipids, amines, sugars, vitamins, co-factors, pigments, antibiotics, peptides, carbohydrates, alcohols, polyols, ureas, purines, pyrimidines, cholines, esters, ethers, pterins, folates, biotin, porphyrins, bilirubins, quinones, phenols, furanones, aldehydes, ketones, carbonyls, isoprenoids, sphingolipids, terpenes, terpenoids, sterols, steroids, phosphates, imidazol es, or lactones.

Proteins which can be used to coat the substrates or culture vessels include, but are not limited to, a growth factor, a cytokine, a peptide hormone, an antibody, a protein hormone, an extracellular matrix protein, an epidermal growth factor (EGF), a platelet derived growth factor (PDGF), a fibroblast growth factor (FGF and bFGF), a transforming growth factor (TGF-a and TGF-P 1, 2, & 3), a vascular endothelial growth factor (VEGF), a hepatocyte growth factor (HGF), a keratinocyte growth factor (KGF), a nerve growth factor (NGF), erythropoietin (EPO), an insulin-like growth factors (IGF-I and IGF-11), an interleukin cytokine (IL-la, IL-Ip, IL-2, IL-3, IL-4, IL-5, IL-6, IL-7, IL-8, IL-9, IL-10, IL-11, IL-12, IL-13), an interferon (IFNa, IFN-p, and IFN-y), a tumor necrosis factor (TNFa and TNF-p), a colony stimulating factor (GM-CSF and M-CSF), insulin, parathyroid hormone, fibronectin, collagenase, collagen, elastin, laminin, agrin, nidogen, entactin, coagulation factor XIII A chain, apolipoprotein E, anithrombin III, bone morphogenic protein 1, vitronectin, acidic leucine-rich nuclear phosphoprotein 32 family member A (ANP32A), calcineurin-like phosphoesterase, peptidyl-prolyl cis-trans isomerase, P-enolase, fermitin family homolog 1, microtubule-associated protein RP/EB (MAPRE1), a heat shock protein, LIM and senescent cell antigen-like-containing domain protein 1 (LIMS 1), myosin regulatory protein, profilin-1, glycogen phosphorylase, flavin reductase, mitogen activated protein kinase, protein phosphatase, tubulin, chloride intracellular channel proteins, wings apartlike protein homolog (WAPAL), cell division control proteins, osteopontin, BPI fold containing, elongation factor, plasminogen, aldo-keto reductase, or keratin, or any fragment thereof

In some kit embodiments, the culture vessels containing the ECM or ECM protein, vessels containing any growth medium or growth supplement, and substrates are sterile and packaged to maintain sterility until use. Methods for maintaining and ensuring sterility may adhere to good manufacturing practice (GMP), good tissue practice (GTP), good laboratory practice (GLP), and good distribution practice (GDP) standards. Methods for maintaining and ensuring sterility include but are not limited to high-efficiency particulate air (HEPA) filtration, wet or dry heat, radiation, e.g., X-rays, gamma rays, or UV light, sterilizing agents or fumigants, such as ethylene oxide, nitrogen dioxide, ozone, glutaraldehyde, formaldehyde, peracetic acid, or hydrogen peroxide, aseptic filling of sterile containers, packaging in plastic film or wrap, or vacuum sealing.

The invention is generally disclosed herein using affirmative language to describe the numerous embodiments. The invention also includes embodiments in which subject matter is excluded, in full or in part, such as substances or materials, method steps and conditions, protocols, or procedures.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

### Selective Induction and Tissue Organization of Endothelial Cells Using Shaped Thin Transparent Glass

### Culture of SMS Cells

The following example demonstrates how an endothelial cell population can be rapidly induced to reorganize and form microvessel structures with the application of a thin glass substrate.

SMS cells are cultured, and ECM or ECM protein isolated from the SMS cells as previously described, for example in WO 2017/172638. Briefly, SMS cells are grown in T25 flask using growth medium (37° C. and 5% CO2). The SMS cell population may contain a heterogeneous cell population of undifferentiated SMS cells and SMS derived differentiated cells.

Undifferentiated SMS cells can be isolated by differential centrifugation, removing clumps of cells or differentiated cells at low centrifugation speed followed by centrifuging undifferentiated SMS cells at high speed. Alternatively, the undifferentiated cells may be isolated by filtration, including differential filtration using filters having progressively smaller pore sizes to a pore size of 3-5 μm. The isolated undifferentiated SMS cells are examined under the microscope for homogeneity.

Undifferentiated SMS cells are grown in polypropylene tube (such as the bioreactor tubes: 15 ml provided by the manufacturer Techno Plastic Products AG, TPP).

The following example of growth medium is used: high sugar basal medium (Dulbecco's Modified Eagle Medium (DIEM), [+] 6 g/L D-glucose, [−] sodium pyruvate, [−] L-glutamine, [−] Phenol red), to which 1% GlutaMAX™-I (100×), 10% calf serum, and 5 μg/mL human insulin was added. Alternatively, a medium not containing any calf serum can be used. The cells are suspended occasionally by swirling.

Complete medium is replaced every week by centrifuging the SMS cells at 4200 g for 15 min. The centrifugation may be varied at 3000 g, 3500 g, 4000 g, 4100 g, 4200 g, 4300g, 4500 g, or 5000 g or by centrifugation at a speed that is within a range defined by any two of the aforementioned speeds, and the time adjusted accordingly.

Under these conditions, the volume size of the medium (cell crowdedness) is growth limiting to SMS cells. The SMS cell homogeneity is assessed microscopically and the SMS cell count is estimated by assessing spectroscopically turbidity of the suspension and/or by measuring the size of the pellet after centrifugation at high speed.

The SMS cell growth potential is assessed by inoculating cells into a new tube with growth medium. SMS cells grow mainly as individual cells not as clumps and remain under this condition mainly undifferentiated. The suspension culture is scalable such that increasing the volume of the medium increases the number of cells obtained.

Preparation of ECM from a Culture of SMS Cells

The SMS cell culture medium is switched by centrifuging the cells at high speed (for example, at 4200 g for 15 min) and suspending in a new growth medium.

SMS cells are seeded onto standard 24-well (about 1.9 cm² of surface area per well bottom), 48-well (about 1.1 cm² of surface area), or 96-well (about 0.32 cm² of surface area) plates (polystyrene; physical surface inducers) and grown using a growth medium (37° C. and 5% CO2). Chemical inducers of ECM and ECM protein production can be provided to the medium, including, for example, a hedgehog inhibitor and a TGF/BMP activator, at growth conditions (37° C. and 5% CO2). The complete medium is added or replaced twice weekly.

ECM or ECM protein is decellularized using various methods known in the art. For example, chemical, physical, and enzymatic methods can be employed to decellularize the ECM or ECM protein, ensuring that the ECM or ECM protein scaffold maintains its structural and chemical integrity. In addition, various molecular components of the SMS-derived ECM or ECM protein are enriched or isolated. ECM or ECM protein in various tissues of various organs can be shown to be similar or identical to ECM or ECM protein obtained from in vitro SMS cell culture. Preparations of SMS cell ECM in the original seeding container can be stored at 4-7° C. or −20° C. until later use.

### Preparation of Angiogenesis-Promoting S-25-Supplemented M-25 Medium

To support the plating and proliferation of human endothelial cells, such as HUVECs, the use of M-25 medium and S-25 supplement is suggested. S-25 is an endothelial supplement that has been optimized for angiogenesis applications, improved cell health, and increased growth rates of endothelial cells.

A 50× premade solution of S-2.5 supplement was thawed in a 37° C. water bath or at 4° C. overnight. The thawed S-25 solution was aseptically transferred to a sterile bottle containing M-25 medium at a ratio of 1:50 (20 μL S-25 supplement per 1 mL M-25 medium), although other ratios, such as 1:25 or 1:75 or any ratio in between these two values, can be used without significant effect on cellular growth. Appropriate antibiotics (e.g. penicillin-streptomycin) was additionally supplemented. Supplemented medium was prepared as needed and ideally used immediately. Unused. S-25-supplemented medium was stored in the dark at 4-7° C. and is stable for up to 3 weeks. Supplemented medium should not be frozen.

### Equilibration of ECM-Coated Culture Vessels

The following procedure details equilibration of ECM coated on 24-well plates, Volumes should be adjusted accordingly for different sized multi-well plates or other culture vessels.

A 24-well plate with ECM coating is allowed to come to room temperature. An appropriate volume of unsupplemented M-25 medium (enough for 1.5 mL per coated well) and S-25-supplemented M-25 medium (enough for 0.5 mL per coated well) is allowed to equilibrate at 37° C. in a humidified atmosphere containing 5% CO₂. Under aseptic conditions, each coated well is washed three times in quick succession with 0.5 mL of equilibrated unsupplemented M-25 medium, aspirating the washes in between, and then 0.5 mL of equilibrated S-25-supplemented M-25 medium is added to each coated well. The coated 24-well plate containing S-25-supplemented M-25 medium is then incubated at 37° C. in a humidified atmosphere containing 5% CO₂.

Plating HUVECs onto the ECM

Cryopreserved HUVECs are thawed rapidly in a 37° C. water bath. The cryotube is incubated for less than 90 seconds and the cell suspension should only be marginally thawed. After 90 seconds in the water bath, the cryotube is gently inverted to thaw cells at room temperature. After complete thawing, 20 μL of the cell suspension was used to determine the concentration and viability of the cells. The cell suspension is diluted four-fold with equilibrated S-25-supplemented M-25 medium, and a volume of cell suspension to obtain 5000-10000 cells per cm² was added. For a 24-well plate, this volume is typically 80-160 μL, although the final concentration of the thawed cells should be empirically assessed. The ECM-coated plate is then gently shaken to disperse the cells and placed at 37° C. in a humidified atmosphere containing 5% CO₂.

### Growing Endothelial Cells on ECM-Coated Plate

After seeding HUVECs onto the ECM and incubating at 37° C. overnight, the medium in each well was replaced with fresh S-25-supplemented medium. To avoid disturbing the cells and ECM layer, the following progression was performed:

(1) 0.25 mL of original medium (50% of the total volume) was removed, and 0.5 mL of fresh supplemented medium was added.

(2) 0.25 mL of medium was removed, and 0.5 mL of fresh supplemental medium was added.

(3) 0.5 mL of medium was removed, and 0.5 mL of fresh supplemental medium was added.

The last step (3) should be repeated three times a week (every 2-3 days) to maintain optimal endothelial cell growth until desired confluency is achieved.

### Endothelial Cell Reorganization and Microvessel Induction Using a Thin Glass Coverslip Substrate

Standard rectangular or circular coverslips of compatible size to fit in the ECM-coated 24-well plate were sterilized and aseptically manipulated with sterile forceps. One or more coverslips were used per contiguous ECM. surface provided that they fit.

The coverslip was placed into the well of the plate containing HUVECs grown on the ECM coating. The coverslip sunk to the bottom of the well and covered a subpopulation of the endothelial cells. This application can be done as soon as the endothelial cells are attached to the bottom of the well or after the cells are allowed to recover, migrate, and proliferate. The medium should be replaced according to the regular schedule described above.

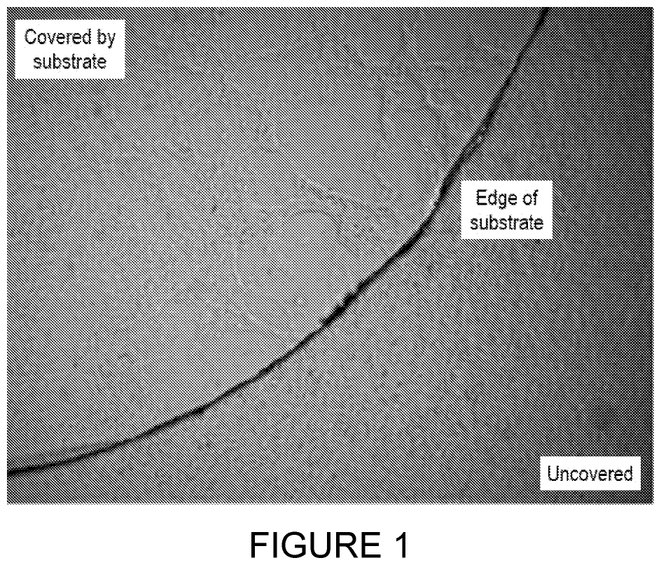

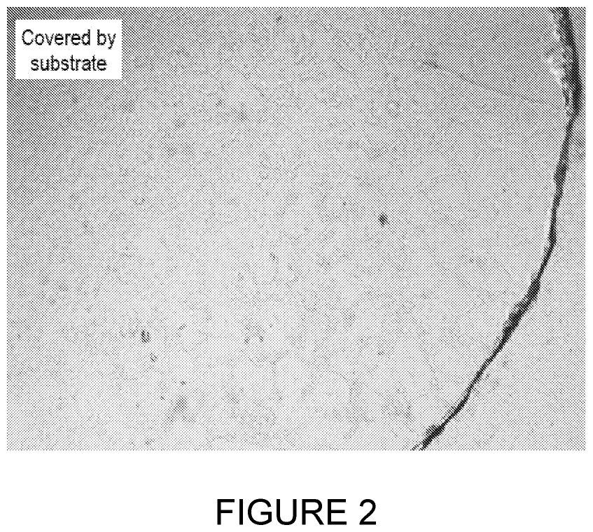

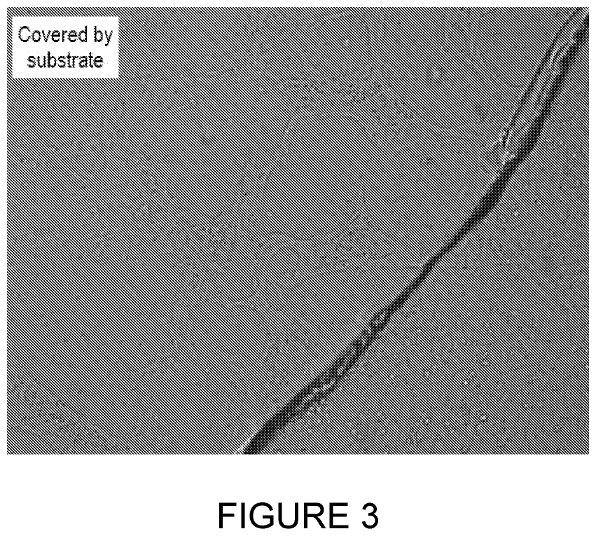

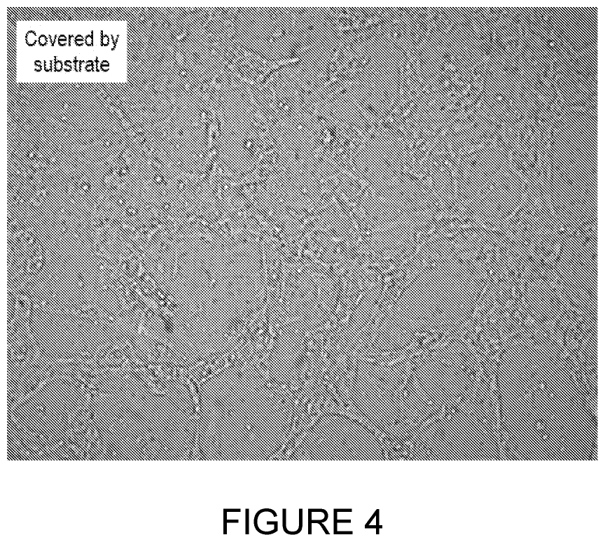

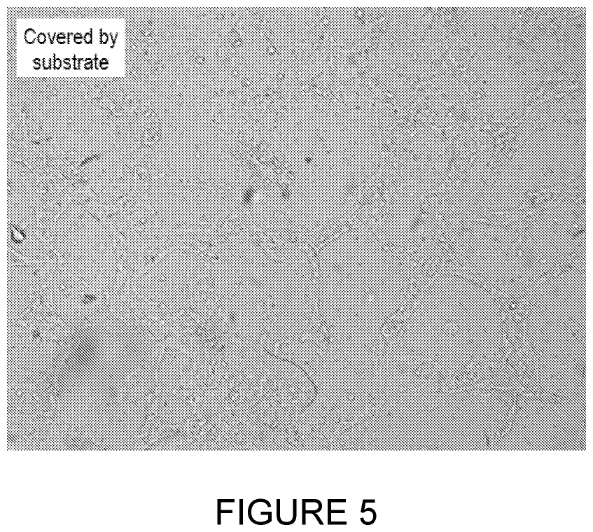

The process of polygonal microvessel organization begins within hours and is typically completed within 2-3 days depending on the substrate size. Reorganization starts at the edges of the coverslip and proceeds inwards between the surfaces of the 24-well plate and coverslip (FIGS. 1 and 2). Cells situated underneath the substrate differentiate to become flat and vacuolized (FIG. 3). Concurrently, these cells migrate to reorganize into a network containing polygonal voids between differentiated endothelial cells reminiscent of microvessel capillaries (FIGS. 4 and 5). This activity can be controlled with the size and shape of the substrate. No special media is required to induce this phenomenon other than the one used for endothelial cell growth.

### Endothelial Cell Reorganization is not Limited By Substrate Shape

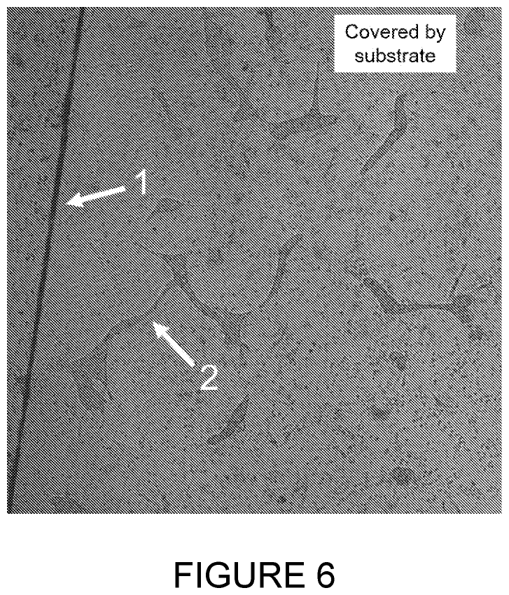

Substrate or coverslip shape is not limited to regular (rectangular, circular) shapes. Glass coverslips can be cracked into small irregular shaped substrates applied in the same manner as described above. Regardless of the shape of the substrate applied to the ECM surface, endothelial cell reorganization consistently initiates at the uncovered and covered cell layer boundary and proceeds inward the substrate (FIG. 6). The shape and size of the substrate can be expanded to three dimensional objects that isolate, partially isolate, occlude, or partially occlude a population of endothelial cells to induce reorganization and microvessel formation. These coverslips and other substrates can also be coated with different substances, molecules, compounds, peptides, or proteins in patterns, shapes, or micropatterns, or altered to feature different physical properties such as stiffness, to engineer complex structural arrangements in the initially uniform endothelial cell population.

### Effect of Different Surface Area Ratios of Substrate to Culture Surface on Endothelial Cell Response

Small round coverslips were used to test differential responses of vascular endothelial cells based on ECM surface size or surface area ratio of coverslip to well. Table 1 shows that a surface area ratio of coverslip to well between 2%-60% induces endothelial cell reorganization. Ratios above 60% show reduced reorganization. This surface area ratio or percentage is applicable to other substrates and culture vessel sizes and shapes.

TABLE 1
Endothelial cell response is dependent on surface area ratio

| | Plate well | Coverslip | Surface area | |
|---|---|---|---|---|
| | surface | surface | ratio | Endothelial |
| | area | area | (coverslip:plate | Cell |
| Plate | (cm²) | (cm²) | well) | response |
| 24 well plate | 1.9 | 0.196 | 10.3% | positive |
| 24 well plate | 1.9 | 0.126 | 6.6% | positive |
| 24 well plate | 1.9 | 0.0707 | 3.7% | positive |
| 48 well plate | 1.1 | 0.196 | 17.8% | positive |
| 48 well plate | 1.1 | 0.126 | 11.5% | positive |
| 48 well plate | 1.1 | 0.0707 | 6.4% | positive |
| 96 well plate | 0.32 | 0.196 | 61.2% | negative |
| 96 well plate | 0.32 | 0.126 | 39.4% | positive |
| 96 well plate | 0.32 | 0.0707 | 22.1% | positive |

By these experiments, it was determined that at least a substrate:well surface area ratio or percentage of 3.7%-39.4% provided a positive endothelial cell response and concomitant tubule or vessel formation but that once the ratio or percentage approaches 61.2% a negative endothelial cell response and negative tubule or vessel formation was identified.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations.” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at leak one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of inducing or accelerating reorganization or differentiation of a population of cells comprising endothelial cells, comprising:
contacting an extracellular matrix (ECM) or ECM protein with said population of cells comprising endothelial cells in a culture vessel; and
adding one or more substrates, which are configured to fit within said culture vessel, to said population of cells comprising endothelial cells in contact with the ECM or ECM protein in the culture vessel, wherein the one or more substrates isolate or occlude or partially isolate or partially occlude the population of cells comprising endothelial cells from growth media or external conditions or both.

2. The method of claim 1, wherein the population of cells comprising endothelial cells further comprises stem cells, fibroblasts, keratinocytes, progenitor cells, neurons, karyocytes, myoblasts, myocytes, adipocytes, osteoblasts, osteocytes, osteoclasts, macrophages, or leukocytes or any combination thereof.

3. The method of claim 1, wherein the ECM is comprised of agrin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, tenascin, perlecan, sortilin-related receptor, tensin, titin, or total protein, or any combination thereof or a fragment of any one or more of the aforementioned.

4. (canceled)

5. The method of claim 1 any one of claims 1, wherein the culture vessel is a dish, plate, well, flask, bottle, chamber, channel, tube, niche, bioreactor, or a container configured to support cell culture.

6. The method of claim 1, wherein the one or more substrates contacting the ECM or ECM protein in the culture vessel contact 2%- 60% of the total available surface area of the ECM or ECM protein in the culture vessel.

7. The method of claim 1, wherein the ratio or percentage of surface area of the substrate to the surface area of the ECM of ECM protein in the culture vessel is between 3.7%-39.4%.

8. The method of claim 1, wherein the population of cells comprising endothelial cells are contacted with the ECM in a growth medium suitable for angiogenesis, arteriogenesis, or vessel formation.

9. The method of claim 8, wherein the growth medium is M-25 medium supplemented with S-25 endothelial cell growth supplement at a concentration of 25:1 to 75:1.

10. The method of claim 1, wherein the endothelial cells are primary endothelial cells or immortalized endothelial cells.

11. The method of claim 1, wherein the endothelial cells are derived or obtained from a human.

12. (canceled)

13. The method of claim 1, wherein the population of cells are grown at 37° C. and in a humidified atmosphere comprised of 5% CO₂.

14. The method of claim 1, wherein the population of cells comprising endothelial cells are grown to 20%, 30%, 40%, 50%, 60%, 70%, 80%, 90%, or 100% confluency.

15. The method of claim 1, wherein the population of cells comprising endothelial cells contacting the one or more substrates reorganize to form microvessel capillary networks or tubule networks.

16. The method of claim 1, wherein the population of cells comprising endothelial cells are allowed to grow in contact with the ECM for 0, 1, 2, 3, 4, 5, 6, 7, 8, 9, 10, 11, 12, 13, 14, 15, 16, 17, 18, 19, 20, 21, 22, 23, 24, 36, or 48 hours or 3-12 days before adding the one or more substrates.

17. The method of claim 1, wherein the population of cells comprising endothelial cells in contact with one or more substrates form tubules or vessel-like structures 0, 1, 2, 3, 4, 5, 6, 7, 8, 9, 10, 11, 12, 13, 14, 15, 16, 17, 18, 19, 20, 21, 22, 23, 24, 36, or 48 hours or 3-12 days after adding the one or more substrates.

18. The method of claim 1, wherein the population of cells comprising endothelial cells in contact with the one or more substrates undergo 80%, 85%, 90%, 95%, or 100% reorganization within 1-5 days.

19. The method of claim 1, wherein the one or more substrates comprise a metal, mineral, plastic, polymer, glass, or ceramic or any combination thereof.

20. The method of claim 1, wherein the ECM or ECM protein is produced from SMS stem cells.

21. -46. (canceled)

47. The method of claim 1, wherein the one or more substrates comprise borosilicate glass.
